(19) United States
(12) Patent Application Publication
(10) Pub. No.: US 2008/0102211 A1
(43) Pub. Date: May 1, 2008

(54) Polysilazane-Treating Solvent and Method for Treating Polysilazane by Using Such Solvent

(76) Inventors: Hideki Matsuo, Shizuoka (JP); Masaaki Ichiyama, Shizuoka (JP); Tomonori Ishikawa, Shizuoka (JP); Hiroyuki Aoki, Tokyo (JP); Bruce Kiker, Colleyville, TX (US); Joseph Oberlander, Phillipsburg, NJ (US)

Correspondence Address: AZ ELECTRONIC MATERIALS USA CORP.;ATTENTION: INDUSTRIAL PROPERTY DEPT., 70 MEISTER AVENUE, SOMERVILLE, NJ, 08876, US

(21) Appl. No.: 11/795,100

(22) PCT Filed: Feb. 1, 2006
(86) PCT No.: PCT/JP06/01662

§ 371 Date: Jul. 10, 2007

(30) Foreign Application Priority Data
Feb. 2, 2005 (JP) 2005-026818

(51) Int. Cl. B05D 3/10; C07C 13/20; C07C 13/19; C07D 493/08; C07C 15/02

(52) U.S. Cl. 427/340; 585/23; 252/364; 585/24; 549/397; 585/26

(57) ABSTRACT

The present invention provides a polysilazane-treating solvent which has excellent dissolving power and stability, has no influence on a substrate as an underlying layer and the properties of a polysilazane, is excellent in shape of cut edge, and further has high safety to the human body. The treating solvent comprises a solvent selected from the group consisting of tetralin, p-menthane, p-cymene, α-pinene, 1,8-cineol, and mixtures thereof, and a polysilazane treatment method using the same. This solvent may further comprise a solvent selected from the group consisting of aliphatic hydrocarbons, alicyclic hydrocarbons, and mixtures thereof.

### TECHNICAL FIELD

The present invention relates to a polysilazane-treating solvent suitable for use in the treatment of a polysilazane coating film or a polysilazane film or the like formed on a base material, and a treatment method for treating a polysilazane compound or a polysilazane coating film using this solvent. More particularly, the present invention provides polysilazane-treating solvent and treatment method that can be suitably used for edge bead removal treatment in which, after the formation of a polysilazane coating film on a substrate, the coating film in its edge part is treated.

### BACKGROUND ART

It has been well known that siliceous films are utilizable as insulating films, dielectric films, protective films, hydrophilized films and the like. Such siliceous films have been formed on base materials by various methods, for example, a PVD method (such as a sputtering method), a CVD method, a sol-gel method, and a method in which a polysiloxane or polysilazane coating film is formed and the coating film is converted to a siliceous film by firing or the like. Among these methods, the PVD and CVD methods suffer from a problem that the apparatus is expensive and, further, very complicated control is necessary for the formation of a good coating film. The sol-gel method is disadvantageous in that the necessary firing temperature is as high as 500° C. or above. Further, the method using polysiloxane suffer from problems such as the occurrence of cracking, for example, due to a reduction in thickness of the formed film. On the other hand, the method in which a solution of a polysilazane compound (various polysilazane compounds being hereinafter often collectively referred to simply as “polysilazane”) is coated and the coating film is converted to a siliceous film, has recently drawn particular attention as a method that a siliceous film having excellent properties can be simply formed by low-temperature firing and the formed siliceous film has excellent properties.

Such siliceous films have extensively utilized, for example, as interlayer insulation films, flattening films, passivation films, and inter-element isolation insulators, for example, in semiconductor elements such as LSIs and TFT liquid crystal display devices. The following method has generally been adopted for the formation of the above siliceous film in semiconductor elements and the like. Specifically, at the outset, a polysilazane solution is spin coated onto a substrate which has or does not have a level difference and is optionally provided with a semiconductor, wiring, an electrode and the like. The coating is then heated to remove the solvent from the coating film. The coating is then fired at a temperature of 350° C. or above to convert the polysilazane to a siliceous film. The converted siliceous film is utilized as inter-layer insulation films, flattening films, passivation films, inter-element isolation insulators and the like. It is well known that this method, however, is disadvantageous in that, when the polysilazane solution is spin coated onto a substrate, beads are formed on the periphery of the substrate and, in addition, the solution sneaks to the backside of the substrate. In order to prevent the coating film from becoming nonuniform in thickness at the peripheral part of the substrate due to the presence of the beads, in general, edge bead removal treatment (hereinafter referred to as “EBR treatment”) is carried out in which, after coating of the polysilazane solution, a treating solvent is coated or sprayed onto the peripheral part of the polysilazane coating film formed on the surface side of the substrate to remove (edge cut) the polysilazane coating film at its peripheral part. In addition, back rinsing is also carried out for removing the polysilazane which sneaked and was deposited onto the backside of the substrate to clean the backside.

Further, the polysilazane film formed by coating using the above method should sometimes be separated from the substrate depending upon the necessity of conducting subsequent treatment, and, further, the polysilazane deposited onto the coating device such as a spin coater should be washed away.

It is known that, for example, propylene glycol monomethyl ether acetate (PGMEA) is used as a rinsing or peeling liquid for removing the polysilazane. The use of the conventional rinsing or peeling liquid, however, is disadvantageous in that, in some cases, the polysilazane cannot be satisfactorily rinsed or peeled, or, even though satisfactory rinsing or peeling of the polysilazane can be realized, clogging of a waste solution line in a coating device such as a spin coater due to gelation of a waste solution or evolution of gas such as silane, hydrogen or ammonia in a waste solution tank occurs. When the gelation of the waste solution occurs, the coating device and waste solution line should be frequently cleaned. Further, when silane gas or the like is evolved in a waste solution tank and the concentration of silane exceeds the spontaneous ignitability limit, for example, a very dangerous state occurs in which explosion occurs at the moment when the lid of the waste solution tank is opened.

The siliceous film formed from the polysilazane coating film is used in various filelds, for example, not only semiconductor elements, but also as dielectric films, insulating films, or partition wall films in liquid crystal display devices and plasma display panels (PDPs), and, further, as protective films on the surface of the body of automobiles and the like, interior and exterior decorating of houses, and various articles such as glass products, ceramic wares, and plastic products. Also in these fields, as with the case of the production of semiconductor elements, in some cases, the polysilazane film deposited on the unnecessry part should be disadvantageously removed.

In view of the above, polysilazane-treating solvents capable of reducing the gelation of the waste solution and the amount of gas evolved have been studied. For example, patent document 1 discloses a polysilazane-treating comprising at least one solvent or a mixture of two or more solvents selected from xylene, anisole, decalin, cyclohexane, cyclohexene, methylcyclohexane, ethylcyclohexane, limonene, hexane, octane, nonane, decane, a C8-C11 alkane mixture, a C8-C11 aromatic hydrocarbon mixture, an aliphatic hydrocarbon/alicyclic hydrocarbon mixture containing not less than 5% by weight and not more than 25% by weight of a C8 or higher aromatic hydrocarbon, and dibutyl ether.

However, from the viewpoint of meeting demands for higher-accuracy quality control, reliable safety and the like in recent years, treating solvents which can further reduce the gelation of the waste solution and the amount of gas evolved as compared with the prior art technique have become required. Further, solvents free from any compound having strong toxicity against the human body, such as naphthalene, trimethylbenzene, and xylene have also been desired. Furthermore, treating solvents, in which the solubility of polysilazanes is high and which has no significant influence on polysilazanes and a substrate as an underlying layer and the like, have been desired. Although such treating solvents have also been used in EBR treatment, in an edge cut part created in the EBR treatment, in some cases, a film thickness increased part called a hump occurs between the film and the film removed part. This hump is causative of cracking or film peeling at the time of firing of the film. For the above reason, treating solvents which can provide films having a better edge cut part shape after the EBR treatment have been desired.

### Patent document 1: Japanese Patent Laid-Open No. 197611/2003

### Patent document 2 Japanese Patent Laid-Open No. 105185/1999

### DISCLOSURE OF THE INVENTION

### Problems to be Solved by the Invention

An object of the present invention is to provide a polysilazane-treating solvent, which can realize EBR treatment in the formation of a polysilazane coating film or rinsing, peeling and the like of a polysilazane film or the like without posing the above-described problems, and a polysilazane compound or polysilazane coating film treatment method using the polysilazane-treating solvent.

That is, an object of the present invention is to provide a polysilazane-treating solvent, which can provide a good edge cut part shape in EBR treatment and hardly decomposes polysilazane, and a polysilazane treatment method using this solvent.

Another object of the present invention is to provide a polysilazane-treating solvent, which, in addition to the above properties, has excellent solubility for polysilazane and has no influence on properties of an underlying semiconductor or substrate or the like and properties of the residual polysilazane coating film, and a polysilazane treatment method using this solvent.

### Means for Solving the Problems

The first polysilazane-treating solvent according to the present invention is characterized by comprising a solvent selected from group 1 consisting of tetralin, p-menthane, p-cymene, α-pinene, 1,8-cineol, and mixtures thereof.

The second polysilazane-treating solvent according to the present invention is characterized by comprising: a solvent selected from group 1 consisting of tetralin, p-menthane, p-cymene, α-pinene, 1,8-cineol, and mixtures thereof; and a solvent selected from group 2 consisting of aliphatic hydrocarbons, alicyclic hydrocarbons, and mixtures thereof.

The first method for treating a polysilazane compound according to the present invention is characterized by comprising bringing an above-mentioned polysilazane-treating solvent into contact with a polysilazane compound.

The second method for treating a polysilazane coating film according to the present invention is characterized by coating a polysilazane compound onto a substrate and then spraying an above-mentioned polysilazane-treating solvent onto an edge part of the polysilazane coating film formed on the substrate or onto the backside of the substrate where no polysilazane coating film was formed, thereby treating the polysilazane coating film.

### Effect of the Invention

The present invention provides a treating solvent which can provide a good edge cut part shape upon EBR treatment. This treating solvent simultaneously has all of properties including excellent solubility for polysilazane, excellent stability in the form of a mixture with polysilazane, and no influence on a substrate as an underlying layer and properties of the polysilazane compound or coating film and thus can also be suitably used for back rinsing when a polysilazane coating liquid was coated onto a substrate. Further, the treating solvent according to the present invention is highly safe to the human body.

### BRIEF DESCRIPTION OF THE DRAWING

FIG. 1 is a cross-sectional view showing a substrate surface after treatment with a treating solvent.

### DESCRIPTION OF REFERENCE CHARACTERS

1: substrate,

2: polysilazane coating film,

3: edge cut part, and

4: raised film thickness.

### BEST MODE FOR CARRYING OUT THE INVENTION

The present invention will be described in more detail.

The polysilazane-treating solvent in the present invention comprises a solvent selected from group 1 consisting of tetralin, p-menthane, p-cymene, α-pinene, 1,8-cineol, and mixtures thereof. When solvents selected from this group are used as a mixture, the mixing ratio is not particularly limited.

Another polysilazane-treating solvent in the present invention comprises: a solvent selected from group 1 consisting of tetralin, p-menthane, p-cymene, α-pinene, 1,8-cineol, and mixtures thereof; and a solvent selected from group 2 consisting of aliphatic hydrocarbons, alicyclic hydrocarbons, and mixtures thereof. Specific examples of solvents belonging to the group 2 include hexane, octane, nonane, decane, decalin, undecane, dodecane, tridecane, tetradecane, isononane, isodecane, isoundecane, isododecane, isotridecane, isotetradecane, cyclononane, cyclodecane, cycloundecane, cyclododecane, cyclotridecane, and cyclotetradecane. A mixture composed of an aliphatic hydrocarbon and an alicyclic hydrocarbon used as solvents belonging to the group 2 may also be mentioned. Such solvents include, for example, EXXSOL D-60 and D-80 (tradenames) commercially available from Exxon Mobil Corporation which may be used in the present invention. The group 1 solvent/group 2 solvent mixing ratio is not particularly limited. Preferably, however, the content of the solvent belonging to group 1 is not less than 10% by weight, more preferably not less than 20% by weight, based on the weight of the whole treating solvent. In general, a higher mixing ratio of the solvent belonging to group 1 is likely to provide better solubility and a better edge cut part shape and thus is preferred. However, the incorporation of the solvent belonging to group 2 is also preferred from the viewpoint of safety because the solvent belonging to group 2 can regulate the flash point, and from the viewpoint of cost effectiveness because the solvent belonging to group 1 is generally expensive.

Among them, the following solvents are particularly preferred because the edge cut part shape which is particularly important in the EBR treatment of semiconductor substrates is likely to be good.

### (1) A treating solvent selected from the group consisting of tetralin, p-menthane, p-cymene, and their mixtures.

(2) A treating solvent comprising: tetralin; and a single or mixed solvent selected from the group consisting of α-pinene, 1,8-cineol, hexane, octane, nonane, decane, decalin, undecane, dodecane, tridecane, tetradecane, isononane, isodecane, isoundecane, isododecane, isotridecane, isotetradecane, cyclononane, cyclodecane, cycloundecane, cyclododecane, cyclotridecane, and cyclotetradecane.
(3) A treating solvent comprising: p-menthane; and a single or mixed solvent selected from the group consisting of α-pinene, 1,8-cineol, hexane, octane, nonane, decane, decalin, undecane, dodecane, tridecane, tetradecane, isononane, isodecane, isoundecane, isododecane, isotridecane, isotetradecane, cyclononane, cyclodecane, cycloundecane, cyclododecane, cyclotridecane, and cyclotetradecane.

The number of fine particles having a size of not less than 0.5 micron contained in 1 ml of the treating solvent according to the present invention is preferably not more than 50, more preferably not more than 10. When the number of fine particles having a size of not less than 0.5 micron contained in 1 ml of the treating solvent exceeds 50, the number of fine particles can be reduced by removing fine particles contained in the treating solvent by proper means such as filtration and distillation to bring the number of fine particles having a size of not less than 0.5 micron contained in 1 ml of the solvent to not more than 50. When the number of fine particles having a size of not less than 0.5 micron contained in 1 ml of the treating solvent exceeds 50, fine particles sometimes stay in the treated polysilazane film. In this case, upon firing of the polysilazane film to form a siliceous film, for example, insulation properties and dielectric properties are sometimes deteriorated. Further, fine particles are adhered onto a semiconductor substrate coated with polysilazane and the like, and this phenomenon sometimes poses a problem such as a deterioration in semiconducting properties and, in some cases, a deterioration in device yield, for example, due to short-circuiting or defective continuity. In particular, the problems of insulation properties, dielectric properties, seimconducting properties, and short-circuiting are frequently observed in the case where the fine particles are metallic. Therefore, the content of metallic fine particles is preferably 0 (zero). More preferably, the number of particles having a size of not less than 0.2 μm is not more than 400.

In the treating solvent according to the present invention, other solvent may be mixed in such an amount that is not detrimental to the effect of the present invention. For example, aromatic hydrocarbons has the effect of enhancing the solubility of polysilazane and thus may be added to enhance the solubility of polysilazane in the treating solvent. Specific examples of such solvents include aromatic hydrocarbon solvents, that is, C8-C11 aromatic hydrocarbon mixtures (for example, Solvesso 100 (tradename) and Solvesso 150 (tradename). available form Shell Oil Corporation, USA, respectively. or aliphatic hydrocarbon/alicyclic hydrocarbon mixtures containing not less than 5% by weight and not more than 25% by weight of a C8 or higher aromatic hydrocarbon (for example, Pegasol AN45 (tradename), available from Exxon Mobil Corporation, USA). However, care should be taken when solvents such as naphthalene, trimethylbenzene, and xylene which are toxic to the human body are used. The amount of such other solvents added is preferably not more than 50% by weight, more preferably not more than 20% by weight, based on the treating solvent according to the present invention.

The treating solvent according to the present invention may be diluted with a mineral spirit as a diluent solvent in such an amount that the object of the present invention can be attained. Also in this case, preferably, the number of fine particles having a size of not less than 0.5 micron contained in 1 ml of the mineral spirit used is not more than 50. When odorless nature is required, solvents which do not give off any significant odor, such as methylcyclohexane and ethylcyclohexane may be selected. Pegasol AN45 (tradename) is a fraction obtained by hydrotreating a distillate oil obtained by atmospheric distillation of a crude oil and is a petroleum hydrocarbon, composed mainly of C8-C11 petroleum hydrocarbons, which is a liquid having an aniline point of 43° C.

Preferably, the treating solvent according to the present invention has a water content of not more than 100 ppm, more preferably not more than 80 ppm. When the water content is not more than 100 ppm, the speed of gelation by the decomposition of polysilazane which comes into contact with the solvent is likely to be decreased, and, for example, the occurrence of problems such as clogging of the waste solution line in the spin coater and the necessity of a long period of time for removing polysilazane deposited onto the spin coater or the like can be advantageously prevented. The prevention of contact between the polysilazane removed from the substrate and water in the waste solution tank can prevent the evolution of gas such as silane, hydrogen, or ammonia. This can reduce the fear of causing the concentration of silane to exceed the spontaneous ignitability limit and can prevent the worst-case scenario such as explosion of the waste solution tank.

The treating solvent according to the present invention can generally be applied to any polysilazane. In this connection, however, it should be noted that the optimal treating solvent varies depending upon the type of polysilazane, that is, depending upon the structure or composition of the polysilazane to be treated. Even when the treating solvent is identical, the solubility of the polysilazane in the treating solvent varies depending on various conditions, for example, whether the polysilazane is inorganic or organic, whether the polysilazane is a homopolymer or a copolymer, the kind of the comonomer in the copolymer, whether or not a cyclic structure is present in the polymer, whether or not the polysilazane is further chemically modified, and whether or not additives are separately added. Further, even when an identical polysilazane is used, the solubility of the polysilazane varies depending upon the type of the solvent. Accordingly, an optimal solvent may be properly selected from the solvents according to the present invention according to the structure or composition of the polysilazane to be treated.

On the other hand, the polysilazane to which the treating solvent according to the present invention is applied may be either inorganic or organic. Among these polysilazanes, inorganic polysilazanes include, for example, perhydropolysilazanes, which comprise a linear structure having a structural unit represented by general formula:

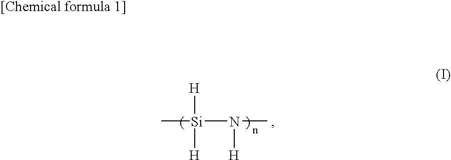

have a molecular weight of 690 to 2000, contain 3 to 10 SiH₃ groups per molecule, and have such elemental proportions as determined by chemical analysis that Si: 59 to 61% by weight, N: 31 to 34% by weight and H: 6.5 to 7.5% by weight, and perhydropolysilazanes having an average molecular weight in the range of 3,000 to 20,000 in terms of polystyrene.

These perhydropolysilazanes may be those which may be produced by any desired method, basically comprise a chain part and a cyclic part in its molecule, and may be represented by chemical formula

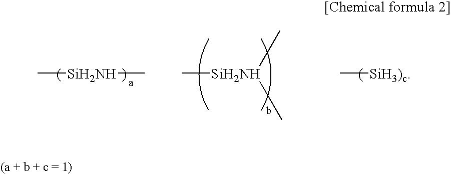

### An example of the perhydropolysilazane structure is as follows.

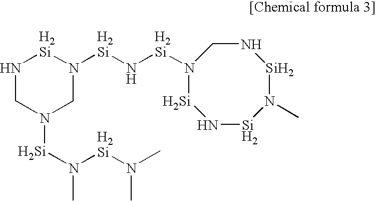

Examples of other polysilazanes include those that have a skeleton, composed mainly of structural units represented by general formula:

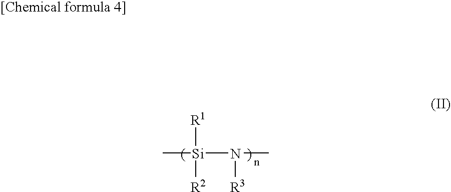

wherein R¹, R², and R³ each independently represent a hydrogen atom, an alkyl group, an alkenyl group, a cycloalkyl group, an aryl group, or a group, which is other than these groups and in which the group connected directly to silicon, such as a fluoroalkyl group is carbon, an alkylsilyl group, an alkylamino group, or an alkoxy group, provided that at least one of R¹, R², and R³ is a hydrogen atom, and a number average molecular weight of about 100 to 50,000, or modification products thereof.

Examples of polysilazanes to which the polysilazane treating solvents according to the present invention is applicable include polyorgano(hydro)silazanes represented by general formula (II) wherein R¹ and R² represent a hydrogen atom and R³ represents an organic group, polysilazanes which have a cyclic structure comprising -(R²SiHNH)— as repeating units and mainly having a degree of polymerization of 3 to 5, polysilazanes which are represented by chemical formula (R³SiHNH)ₓ[(R²SiH)_{1.5}N]₁₋ₓ wherein 0.4<X<1 and simultaneously have, in the molecule thereof, a chain structure and a cyclic structure, polysilazanes represented by general formula (II) wherein R¹ represents a hydrogen atom and R² and R³ represent an organic group, and polysilazanes which have a cyclic structure comprising, as repeating units, -(R¹R²SiNR³)— wherein R¹ and R² represent an organic group and R³ represents a hydrogen atom, and mainly having a degree of polymerization of 3 to 5.

Organic polysilazanes other than those represented by general formula (II) include, for example, polyorgano(hydro)silazanes having, in the molecule thereof, a crosslinked structure represented by general formula:

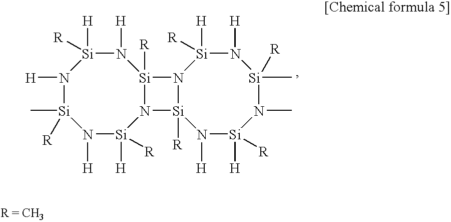

polysilazanes having a crosslinked structure formed by ammonolysis of R¹SiX₃ (X: halogen), and polysilazanes having the following structure prepared by co-ammonolysis of R¹Si(NH)ₓ or R¹SiX₃ and R²₂SiX₂.

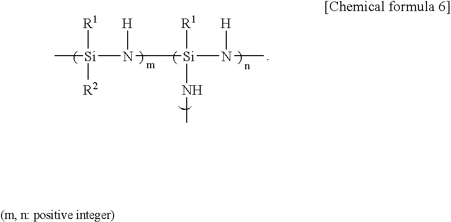

Other polysilazanes include polysiloxazanes comprising repeating units represented by [(SiH₂)ₙ(NH)ₘ] and [(SiH₂)ᵣO] wherein n, m, and r are each 1, 2, or 3, modified polysilazanes prepared by adding an alcohol such as methanol or hexamethyldisilazane to the terminal N atom of perhydropolysilazanes, and metal-containing polysilazanes containing a metal, for example, aluminum.

Still other polysilazanes include polysilazanes, for example, polyborosilazanes, inorganic silazane high polymers and modified polysilazanes, interpolysilazanes, polysilazanes which have been rendered ceramic at a low temperature and to which a catalytic compound for accelerating the conversion of the polysilazane to ceramic has been added or mixed, polysilazanes with a silicon alkoxide added thereto, polysilazanes with glycidol added thereto, polysilazanes with an acetylacetonate complex added thereto, and polysilazanes with a metal carboxylate added thereto, and polysilazane compositions prepared by adding amines or/and acids to the above various polysilazanes or modification products thereof.

The form of a polysilazane to which the solvent of the present invention is applied is generally a film but is not limited to the film. The polysilazane may be covered on a base material by any method without particular limitation, for example, a conventional method such as spin coating, spray coating, flow coating, roller coating, dip coating, cloth wiping, or sponge wiping. Further, the base material may also be in any form such as a plate or a film, and the surface may be in a flat or concave-convex form or in a curved surface form. The base material may be formed of any of a semiconductor, glass, a metal, a metal oxide, a plastic and the like.

The solvent according to the present invention may be brought into contact with the polysilazane by any method without particular limitation, and examples thereof include a method in which the solvent is sprayed or jetted through a nozzle onto the polysilazane on the base material, a method in which the base material with the polysilazane coated thereon is immersed in the solvent, and a method in which the polysilazane is washed away with the solvent.

The method for conducting EBR treatment using the solvent according to the present invention will be described, for example, by taking, as an example, the case where a polysilazane solution is coated onto a semiconductor substrate (a silicon wafer) to form, for example, an inter-layer insulation film, a flattening film, a passivation film, or an inter-element isolation film on the semiconductor substrate. Specifically, an 8-inch silicon wafer optionally provided with a semiconductor, wiring or the like is mounted on a spin coater. A polysilazane solution is spin coated onto the wafer being rotated, for example, at a speed of rotation of 500 to 4000 rpm. Next, in such a state that the polysilazane-coated wafer is rotated, the solvent according to the present invention is sprayed as a washing liquid (a rinsing liquid) through a nozzle onto the edge part of the coating film to bring the solvent into contact with the polysilazane, whereby beads at the edge part of the wafer are removed. When EBR treatment is carried out subsequent to coating of the wafer by a conventional spin coater, the EBR treatment is preferably carried out under the following conditions.

Speed of rotation of coater in EBR treatment: 1000 to 6000 rpm
Flow rate of treating solvent sprayed through nozzle: 2 to 100 ml/min
Pressure under which treating solvent is sprayed through nozzle: 0.01 to 1 MPa
Treating solvent spraying time: 0.01 to 60 sec
In this case, the polysilazane-treating solvent may be simultaneously sprayed onto the backside of the substrate for back rinsing. Although the EBR treatment and the back rinsing may be carried out separately from each other, preferably, the EBR treatment is carried out simultaneously with the back rinsing because the step of back rinsing can be omitted.

### EXAMPLES 1 TO 26 AND COMPARATIVE EXAMPLES 1 TO 7

Treating solvents listed in Table 1 and polysilazanes, that is, perhydropolysilazane, polysilazane with methanol added thereto, and polysilazane with hexamethyldisilazane added thereto described in Reference Examples 1 to 3 of patent document 1, and aluminum-containing polysilazane described in Example 1 of patent document 2 were provided. For the individual polysilazanes, the number of days necessary for gelation, the amount of gas evolved, and properties of edge cut part were evaluated by the following methods.

(Method for Evaluating Number of Days Necessary for Gelation)

A 20 wt % solution (5 g) of each polysilazane compound in di-n-butyl ether and 50 g of the treating solvent were placed and mixed together in a glass bottle (100 g). The glass bottle was allowed to stand with the lid opened in a room under conditions of 22° C. and 50% RH to visually determine the number of days necessary for gelation. In general, the number of days necessary for gelation is preferably two days or longer, more preferably three days or longer.

(Method for Evaluating Amount of Gas Evolved)

A 20 wt % solution (5 g) of each polysilazane compound in di-n-butyl ether and 50 g of the treating solvent were placed and mixed together in a glass bottle (100 g), and the bottle was then hermetically sealed. One hour after the hermetical sealing, the gaseous phase part was sampled for gas chromatographic analysis.

(Method for Evaluating Shape of Edge Cut Part Formed by EBR Treatment, and State of Backside by Back Rinsing)

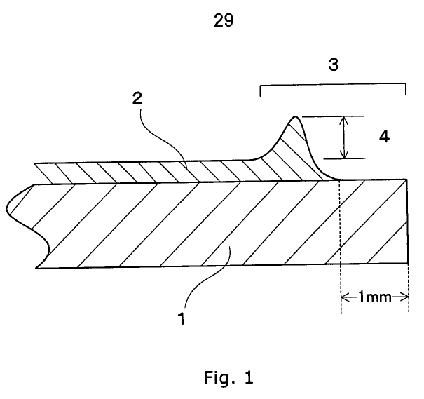

CLEAN TRACK Mark-8, manufactured by Tokyo Electron Limited was provided. A 20 wt % solution of each polysilazane compound in di-n-butyl ether was spin coated at a speed of rotation of 1000 rpm for 10 sec, and, subsequently, the treating solvent was sprayed onto the peripheral part of the coating film on the surface of the substrate and onto the backside of the substrate under conditions of speed of rotation 2000 rpm and 5 sec for EBR treatment simultaneously with back rinsing. For the film formed surface, the treating solvent was sprayed to a position of 1 mm inward from the outer periphery of the wafer, and, for the backside, the treating solvent was sprayed to a position of 3 mm inward from the outer periphery of the wafer. FIG. 1 is a typical cross-sectional view of the treated substrate. The average value of the film thickness was 0.35 μm. A coating film 2 of polysilazane formed on a substrate 1 had a hump at its edge cut part 3. The edge cut shape on the film formed face was measured with Spectral Reflectance Thickness Monitor FE-3000, manufactured by Otsuka Electronics Co., Ltd for evaluation of the raised film thickness 4 in the edge cut part. The level of raised film thickness at the edge cut part is preferably less than 1 μm from the practical point of view. The backside was evaluated by observation under an optical microscope to confirm whether or not there is a residue.

The results thus obtained were as shown in Tables 2 to 5.

TABLE 1

| | | | | | | Water |
|---|---|---|---|---|---|---|
| | | | | | Particles*² | content |
| | Treating Solvent*¹ | | | | (/ml) | (wt %) |
| Ex. 1 | Tetralin | (100) | | | 5 | 0.002 |
| Ex. 2 | p-Menthane | (100) | | | 5 | 0.002 |
| Ex. 3 | p-Cymene | (100) | | | 4 | 0.002 |
| Ex. 4 | α-Pinene | (100) | | | 5 | 0.003 |
| Ex. 5 | Tetralin | (50) | +p-Menthane | (50) | 5 | 0.002 |
| Ex. 6 | Tetralin | (50) | +p-Cymene | (50) | 5 | 0.002 |
| Ex. 7 | Tetralin | (50) | +α-Pinene | (50) | 3 | 0.002 |
| Ex. 8 | Tetralin | (50) | +1,8-Cineol | (50) | 4 | 0.003 |
| Ex. 9 | Tetralin | (50) | +Hexane | (50) | 4 | 0.002 |
| Ex. 10 | Tetralin | (50) | +Octane | (50) | 6 | 0.002 |
| Ex. 11 | Tetralin | (50) | +Nonane | (50) | 5 | 0.002 |
| Ex. 12 | Tetralin | (50) | +Decane | (50) | 4 | 0.002 |
| Ex. 13 | Tetralin | (50) | +Decalin | (50) | 6 | 0.005 |
| Ex. 14 | Tetralin | (50) | +D-60*³ | (50) | 2 | 0.002 |
| Ex. 15 | Tetralin | (20) | +D-80*⁴ | (80) | 2 | 0.002 |
| Ex. 16 | Tetralin | (30) | +D-80 | (70) | 4 | 0.002 |
| Ex. 17 | Tetralin | (50) | +D-80 | (50) | 3 | 0.002 |
| Ex. 18 | p-Menthane | (50) | +α-Pinene | (50) | 2 | 0.002 |
| Ex. 19 | p-Menthane | (50) | +1,8-Cineol | (50) | 4 | 0.003 |
| Ex. 20 | p-Menthane | (50) | +Hexane | (50) | 1 | 0.002 |
| Ex. 21 | p-Menthane | (50) | +Octane | (50) | 2 | 0.002 |
| Ex. 22 | p-Menthane | (50) | +Nonane | (50) | 8 | 0.002 |
| Ex. 23 | p-Menthane | (50) | +Decane | (50) | 4 | 0.002 |
| Ex. 24 | p-Menthane | (50) | +Decalin | (50) | 6 | 0.005 |
| Ex. 25 | p-Menthane | (50) | +D-60 | (50) | 5 | 0.002 |
| Ex. 26 | p-Menthane | (50) | +D-80 | (50) | 3 | 0.002 |
| Comp. | Nonane | (100) | | | 5 | 0.001 |
| Ex. 1 | | | | | | |
| Comp. | Dipentene | (100) | | | 4 | 0.003 |
| Ex. 2 | | | | | | |
| Comp. | D-80 | (100) | | | 3 | 0.004 |
| Ex. 3 | | | | | | |
| Comp. | DBE*⁵ | (100) | | | 3 | 0.005 |
| Ex. 4 | | | | | | |
| Comp. | PGMEA*⁶ | (100) | | | 1 | 0.01 |
| Ex. 5 | | | | | | |
| Comp. | PGME*⁷ | (70) | + PGMEA | (30) | 1 | 0.02 |
| Ex. 6 | | | | | | |
| Comp. | Decalin | (100) | | | 5 | 0.002 |
| Ex. 7 | | | | | | |

Note:
*¹The numerical value within parentheses in the column of “Treating solvent” indicates mixing ratio on weight basis.
*²The number of particles having size of more than 0.5 μm per unit volume in treating solvent
*^{3,4}Aliphatic hydrocarbon/alicyclic hydrocarbon mixture manufactured by Exxon Mobil Corporation: tradename
*⁵Dibutyl ether
*⁶Propylene glycol monomethyl ether acetate
*⁷Propylene glycol monomethyl ether

TABLE 2

| | Perhydropolysilazane | | | | |
|---|---|---|---|---|---|
| | | | | Shape of | |
| | | | | edge cut | |
| | Number of | | | part | |
| | days for | Evolved gas | | (Raised film | Back |
| | gelation | Monosilane | Hydrogen | thickness) | rinsing |
| | (days) | (vol %) | (vol %) | (μm) | (Residue) |
| Ex. 1 | 4 | 0.001 | 0.05 | 0 | Good |
| Ex. 2 | 3 | 0.003 | 0.1 | 0.1 | Good |
| Ex. 3 | 3 | 0.003 | 0.4 | 0.3 | Good |
| Ex. 4 | 5 | 0.003 | 0.05 | 2 | Good |
| Ex. 5 | 3 | 0.003 | 0.1 | 0 | Good |
| Ex. 6 | 3 | 0.003 | 0.1 | 0.1 | Good |
| Ex. 7 | 4 | 0.001 | 0.05 | 0.5 | Good |
| Ex. 8 | 2 | 0.01 | 0.1 | 0.5 | Good |
| Ex. 9 | 2 | 0.005 | 0.05 | 0.4 | Good |
| Ex. 10 | 2 | 0.002 | 0.05 | 0.4 | Good |
| Ex. 11 | 2 | 0.002 | 0.06 | 0.4 | Good |
| Ex. 12 | 2 | 0.002 | 0.1 | 0.4 | Good |
| Ex. 13 | 3 | 0.002 | 0.1 | 0 | Good |
| Ex. 14 | 3 | 0.004 | 0.04 | 0.3 | Good |
| Ex. 15 | 3 | 0.002 | 0.03 | 0.05 | Good |
| Ex. 16 | 3 | 0.002 | 0.02 | 0 | Good |
| Ex. 17 | 3 | 0.002 | 0.02 | 0 | Good |
| Ex. 18 | 4 | 0.003 | 0.02 | 0.6 | Good |
| Ex. 19 | 2 | 0.01 | 0.1 | 0.6 | Good |
| Ex. 20 | 2 | 0.003 | 0.05 | 0.6 | Good |
| Ex. 21 | 2 | 0.003 | 0.05 | 0.6 | Good |
| Ex. 22 | 2 | 0.003 | 0.05 | 0.6 | Good |
| Ex. 23 | 2 | 0.003 | 0.05 | 0.6 | Good |
| Ex. 24 | 3 | 0.003 | 0.03 | 0.7 | Good |
| Ex. 25 | 2 | 0.003 | 0.06 | 0.5 | Good |
| Ex. 26 | 2 | 0.003 | 0.05 | 0.3 | Good |
| Comp. | 1 | 0.004 | 0.07 | Not peelable | Failed |
| Ex. 1 | | | | | |
| Comp. | 1 | 0.005 | 0.06 | 3 | Good |
| Ex. 2 | | | | | |
| Comp. | 1 | 0.003 | 0.05 | 0.3 | Failed |
| Ex. 3 | | | | | |
| Comp. | 1 | 0.008 | 0.1 | 1.5 | Good |
| Ex. 4 | | | | | |
| Comp. | 0.5 | 0.5 | 25 | 1 | Good |
| Ex. 5 | | | | | |
| Comp. | >10 | 1.4 | 25 | 1 | Good |
| Ex. 6 | | | | | |
| Comp. | 2 | 0.003 | 0.03 | Not peelable | Failed |
| Ex. 7 | | | | | |

TABLE 3

| | Polysilazane with methanol added thereto | | | | |
|---|---|---|---|---|---|
| | | | | Shape of | |
| | | | | edge cut | |
| | Number of | | | part | |
| | days for | Evolved gas | | (Raised film | Back |
| | gelation | Monosilane | Hydrogen | thickness) | rinsing |
| | (days) | (vol %) | (vol %) | (μm) | (Residue) |
| Ex. 1 | 5 | 0.002 | 0.1 | 0 | Good |
| Ex. 2 | 4 | 0.003 | 0.08 | 0.1 | Good |
| Ex. 3 | 4 | 0.003 | 0.5 | 0.4 | Good |
| Ex. 4 | 5 | 0.003 | 0.06 | 2 | Good |
| Ex. 5 | 4 | 0.004 | 0.1 | 0.1 | Good |
| Ex. 6 | 4 | 0.005 | 0.1 | 0.1 | Good |
| Ex. 7 | 4 | 0.001 | 0.1 | 0.4 | Good |
| Ex. 8 | 3 | 0.02 | 0.2 | 0.5 | Good |
| Ex. 9 | 3 | 0.01 | 0.1 | 0.5 | Good |
| Ex. 10 | 3 | 0.01 | 0.1 | 0.4 | Good |
| Ex. 11 | 3 | 0.005 | 0.1 | 0.5 | Good |
| Ex. 12 | 3 | 0.002 | 0.1 | 0.4 | Good |
| Ex. 13 | 3 | 0.002 | 0.1 | 0.1 | Good |
| Ex. 14 | 3 | 0.004 | 0.1 | 0.3 | Good |
| Ex. 15 | 3 | 0.003 | 0.1 | 0 | Good |
| Ex. 16 | 3 | 0.003 | 0.2 | 0 | Good |
| Ex. 17 | 3 | 0.003 | 0.2 | 0 | Good |
| Ex. 18 | 4 | 0.003 | 0.05 | 0.6 | Good |
| Ex. 19 | 3 | 0.02 | 0.15 | 0.3 | Good |
| Ex. 20 | 3 | 0.005 | 0.1 | 0.4 | Good |
| Ex. 21 | 3 | 0.004 | 0.1 | 0.4 | Good |
| Ex. 22 | 3 | 0.003 | 0.1 | 0.4 | Good |
| Ex. 23 | 3 | 0.005 | 0.1 | 0.4 | Good |
| Ex. 24 | 3 | 0.004 | 0.05 | 0.5 | Good |
| Ex. 25 | 3 | 0.002 | 0.08 | 0.4 | Good |
| Ex. 26 | 3 | 0.005 | 0.07 | 0.4 | Good |
| Comp. | 1 | 0.005 | 0.07 | Not peelable | Failed |
| Ex. 1 | | | | | |
| Comp. | 1 | 0.006 | 0.08 | 3 | Good |
| Ex. 2 | | | | | |
| Comp. | 1 | 0.005 | 0.03 | 0.3 | Failed |
| Ex. 3 | | | | | |
| Comp. | 1 | 0.009 | 0.1 | 1.5 | Good |
| Ex. 4 | | | | | |
| Comp. | 0.5 | 0.4 | 20 | 1 | Good |
| Ex. 5 | | | | | |
| Comp. | >10 | 1.5 | 25 | 1 | Good |
| Ex. 6 | | | | | |
| Comp. | 2 | 0.04 | 0.04 | Not peelable | Failed |
| Ex. 7 | | | | | |

TABLE 4

| | Polysilazane with hexamethyldisilazane added thereto | | | | |
|---|---|---|---|---|---|
| | | | | Shape of | |
| | | | | edge cut | |
| | Number of | | | part | |
| | days for | Evolved gas | | (Raised film | Back |
| | gelation | Monosilane | Hydrogen | thickness) | rinsing |
| | (days) | (vol %) | (vol %) | (μm) | (Residue) |
| Ex. 1 | 7 | 0.001 | 0.05 | 0 | Good |
| Ex. 2 | 7 | 0.003 | 0.06 | 0.1 | Good |
| Ex. 3 | 7 | 0.02 | 0.1 | 0.2 | Good |
| Ex. 4 | 5 | 0.002 | 0.03 | 1.5 | Good |
| Ex. 5 | 6 | 0.002 | 0.05 | 0 | Good |
| Ex. 6 | 6 | 0.002 | 0.08 | 0.2 | Good |
| Ex. 7 | 6 | 0.001 | 0.04 | 0.4 | Good |
| Ex. 8 | 6 | 0.01 | 0.1 | 0.5 | Good |
| Ex. 9 | 7 | 0.002 | 0.06 | 0.3 | Good |
| Ex. 10 | 7 | 0.002 | 0.05 | 0.4 | Good |
| Ex. 11 | 7 | 0.002 | 0.04 | 0.4 | Good |
| Ex. 12 | 7 | 0.002 | 0.05 | 0.3 | Good |
| Ex. 13 | 7 | 0.002 | 0.05 | 0.1 | Good |
| Ex. 14 | 7 | 0.003 | 0.04 | 0.3 | Good |
| Ex. 15 | 7 | 0.001 | 0.03 | 0.1 | Good |
| Ex. 16 | 7 | 0.001 | 0.01 | 0 | Good |
| Ex. 17 | 7 | 0.003 | 0.01 | 0 | Good |
| Ex. 18 | 7 | 0.003 | 0.02 | 0.6 | Good |
| Ex. 19 | 7 | 0.005 | 0.1 | 0.5 | Good |
| Ex. 20 | 7 | 0.002 | 0.01 | 0.6 | Good |
| Ex. 21 | 7 | 0.002 | 0.03 | 0.5 | Good |
| Ex. 22 | 7 | 0.003 | 0.03 | 0.6 | Good |
| Ex. 23 | 7 | 0.004 | 0.03 | 0.4 | Good |
| Ex. 24 | 7 | 0.003 | 0.04 | 0.7 | Good |
| Ex. 25 | 7 | 0.001 | 0.05 | 0.2 | Good |
| Ex. 26 | 7 | 0.001 | 0.05 | 0.2 | Good |
| Comp. | 1 | 0.005 | 0.07 | Not peelable | Failed |
| Ex. 1 | | | | | |
| Comp. | 1 | 0.005 | 0.08 | 3 | Good |
| Ex. 2 | | | | | |
| Comp. | 1 | 0.005 | 0.05 | 0.3 | Failed |
| Ex. 3 | | | | | |
| Comp. | 2 | 0.008 | 0.09 | 1.5 | Good |
| Ex. 4 | | | | | |
| Comp. | 0.5 | 0.3 | 15 | 1 | Good |
| Ex. 5 | | | | | |
| Comp. | >10 | 1 | 20 | 1 | Good |
| Ex. 6 | | | | | |
| Comp. | 2 | 0.003 | 0.02 | Not peelable | Failed |
| Ex. 7 | | | | | |

TABLE 5

| | Aluminum-containing polysilazane | | | | |
|---|---|---|---|---|---|
| | | | | Shape of | |
| | | | | edge cut | |
| | Number of | | | part | |
| | days for | Evolved gas | | (Raised film | Back |
| | gelation | Monosilane | Hydrogen | thickness) | rinsing |
| | (days) | (vol %) | (vol %) | (μm) | (Residue) |
| Ex. 1 | 3 | 0.001 | 0.05 | 0 | Good |
| Ex. 2 | 2 | 0.003 | 0.1 | 0 | Good |
| Ex. 3 | 2 | 0.003 | 0.4 | 0 | Good |
| Ex. 4 | 3 | 0.003 | 0.05 | 0.15 | Good |
| Ex. 5 | 2 | 0.003 | 0.1 | 0 | Good |
| Ex. 6 | 2 | 0.003 | 0.1 | 0 | Good |
| Ex. 7 | 3 | 0.001 | 0.05 | 0.1 | Good |
| Ex. 8 | 2 | 0.01 | 0.1 | 0.1 | Good |
| Ex. 9 | 1 | 0.005 | 0.05 | 0.1 | Good |
| Ex. 10 | 1 | 0.002 | 0.05 | 0.1 | Good |
| Ex. 11 | 1 | 0.002 | 0.06 | 0.1 | Good |
| Ex. 12 | 1 | 0.002 | 0.1 | 0.1 | Good |
| Ex. 13 | 1 | 0.002 | 0.1 | 0 | Good |
| Ex. 14 | 1 | 0.004 | 0.04 | 0 | Good |
| Ex. 15 | 1 | 0.002 | 0.03 | Not peelable | Failed |
| Ex. 16 | 1 | 0.002 | 0.02 | Not peelable | Failed |
| Ex. 17 | 1 | 0.002 | 0.02 | 0 | Good |
| Ex. 18 | 3 | 0.003 | 0.02 | 0.2 | Good |
| Ex. 19 | 2 | 0.01 | 0.1 | 0.2 | Good |
| Ex. 20 | 1 | 0.003 | 0.05 | 0.2 | Good |
| Ex. 21 | 1 | 0.003 | 0.05 | 0.2 | Good |
| Ex. 22 | 1 | 0.003 | 0.05 | 0.2 | Good |
| Ex. 23 | 1 | 0.003 | 0.05 | 0.2 | Good |
| Ex. 24 | 1 | 0.003 | 0.03 | 0.2 | Good |
| Ex. 25 | 1 | 0.003 | 0.06 | 0.3 | Good |
| Ex. 26 | 1 | 0.003 | 0.05 | 0.3 | Good |
| Comp. | 0 | 0.005 | 0.07 | Not peelable | Failed |
| Ex. 1 | | | | | |
| Comp. | 1 | 0.005 | 0.06 | 1.5 | Good |
| Ex. 2 | | | | | |
| Comp. | 0 | 0.004 | 0.05 | Not peelable | Failed |
| Ex. 3 | | | | | |
| Comp. | 1 | 0.009 | 0.1 | 1 | Good |
| Ex. 4 | | | | | |
| Comp. | 0.5 | 0.6 | 30 | 0.5 | Good |
| Ex. 5 | | | | | |
| Comp. | >10 | 1 | 20 | 0.5 | Good |
| Ex. 6 | | | | | |
| Comp. | 1 | 0.004 | 0.05 | Not peelable | Failed |
| Ex. 7 | | | | | |

The following points are apparent from the results.

When nonane, dipentene, D-80, dibutyl ether or decalin was used as the treating solvent, although the amount of gas evolved was small, the number of days for gelation was small and, further, there is room for improvement in edge cut part shape. When D-80 was used as the treating solvent, a residue was observed on the backside of the substrate, probably due to low solubility for polysilazane.

When a mixed solvent composed of PGMEA or PGME and PGMEA was used as the treating solvent, the amount of gas evolved was large and the edge cut part shape was also unsatisfactory.

On the other hand, the treating solvents according to the present invention are improved in all of the number of days for gelation, the amount of gas evolved, and edge cut part shape, demonstrating that the treating solvents according to the present invention are superior solvents.

1. A polysilazane-treating solvent comprising a solvent selected from group 1 consisting of tetralin, p-cymene, α-pinene, 1,8-cineol, and mixtures thereof.

2. A polysilazane-treating solvent comprising: a solvent selected from group 1 consisting of tetralin, p-cymene, α-pinene, 1,8-cineol, and mixtures thereof; and a solvent selected from group 2 consisting of aliphatic hydrocarbons, alicyclic hydrocarbons, and mixtures thereof.

3. The polysilazane-treating solvent according to claim 1, wherein the number of fine particles having a size of not less than 0.5 micron contained in 1 ml of said treating solvent is not more than 50.

4. The polysilazane-treating solvent according to claim 1, which has a water content of not more than 100 ppm.

5. A method for treating a polysilazane, comprising bringing a polysilazane-treating solvent according to claim 1 into contact with a polysilazane compound.

6. A method for treating a polysilazane, comprising coating a polysilazane compound onto a substrate and then spraying a polysilazane-treating solvent according to claim 1 onto an edge part of the polysilazane coating film formed on the substrate or onto the backside of the substrate where no polysilazane coating film was formed, thereby treating the polysilazane coating film.

7. The polysilazane-treating solvent according to claim 2, wherein the number of fine particles having a size of not less than 0.5 micron contained in 1 ml of said treating solvent is not more than 50.

8. The polysilazane-treating solvent according to claim 2, which has a water content of not more than 100 ppm.

9. The polysilazane-treating solvent according to claim 2 wherein the solvent selected from group 2 is a single solvent or mixed solvent selected from the group consisting of hexane, octane, nonane, decane, decalin, undecane, dodecane, tridecane, tetradecane, isononane, isodecane, isoundecane, isododecane, isotridecane, isotetradecane, cyclononane, cyclodecane, cycloundecane, cyclododecane, cyclotridecane, and cyclotetradecane.

10. The polysilazane-treating solvent according to claim 2 comprising tetralin and a single solvent or mixed solvent selected from the group consisting of α-pinene, 1,8-cineol, hexane, octane, nonane, decane, decalin, undecane, dodecane, tridecane, tetradecane, isononane, isodecane, isoundecane, isododecane, isotridecane, isotetradecane, cyclononane, cyclodecane, cycloundecane, cyclododecane, cyclotridecane, and cyclotetradecane.

11. The polysilazane-treating solvent according to claim 3, which has a water content of not more than 100 ppm.

12. A method for treating a polysilazane, comprising bringing a polysilazane-treating solvent according to claim 2 into contact with a polysilazane compound.

13. A method for treating a polysilazane, comprising bringing a polysilazane-treating solvent according to claim 3 into contact with a polysilazane compound.

14. A method for treating a polysilazane, comprising bringing a polysilazane-treating solvent according to claim 4 into contact with a polysilazane compound.

15. A method for treating a polysilazane, comprising coating a polysilazane compound onto a substrate and then spraying a polysilazane-treating solvent according to claim 2 onto an edge part of the polysilazane coating film formed on the substrate or onto the backside of the substrate where no polysilazane coating film was formed, thereby treating the polysilazane coating film.

16. A method for treating a polysilazane, comprising coating a polysilazane compound onto a substrate and then spraying a polysilazane-treating solvent according to claim 3 onto an edge part of the polysilazane coating film formed on the substrate or onto the backside of the substrate where no polysilazane coating film was formed, thereby treating the polysilazane coating film.

17. A method for treating a polysilazane, comprising coating a polysilazane compound onto a substrate and then spraying a polysilazane-treating solvent according to claim 4 onto an edge part of the polysilazane coating film formed on the substrate or onto the backside of the substrate where no polysilazane coating film was formed, thereby treating the polysilazane coating film.
